(19) United States
(12) Patent Application Publication
(10) Pub. No.: US 2025/0235546 A1
(43) Pub. Date: Jul. 24, 2025

(54) TREATMENT OF CANCER USING A COMBINATION OF IMMUNOMODULATION AND CHECK POINT INHIBITORS

(71) Applicant: ANGIMMUNE, LLC, Bethesda, MD (US)

(72) Inventors: David M. Neville, Bethesda, MD (US); Yuan Yi Liu, Bethesda, MD (US)

(21) Appl. No.: 18/999,360

(22) Filed: Dec. 23, 2024

Related U.S. Application Data
(63) Continuation of application No. 16/271,153, filed on Feb. 8, 2019, now abandoned.
Application No. 16/271,153 is a continuation-in-part of application No. PCT/US2017/045823, filed on Aug. 8, 2017.
(60) Provisional application No. 62/372,444, filed on Aug. 9, 2016.

(51) Int. Cl. A61K 47/64; A61K 39/00; A61K 39/39; A61K 45/06; A61K 47/68; A61N 5/10; A61P 35/00; C07K 16/28

(52) U.S. Cl. CPC A61K 47/6415; A61K 39/39; A61K 45/06; A61K 47/6849; A61N 5/10; A61P 35/00; C07K 16/2809; C07K 16/2818; C07K 16/2827; A61K 2039/505; A61K 2039/507; A61K 2039/585; A61K 2039/876; C07K 2317/24; C07K 2317/622; C07K 2317/76; C07K 2319/55

(57) ABSTRACT

Methods of treating patients suffering from cancer using a combination of 1) an immunomodulatory agent; 2) a check point inhibitor; and 3) a cancer antigen releasing therapy are provided. In some aspects, the immunomodulatory agent is an anti-CD3 immunotoxin, the check point inhibitor inhibits the interaction of PD-1 and PD-L1 and the cancer antigen releasing therapy is radiation.

### SEQUENCE LISTING

This document incorporates by reference an electronic sequence listing file, which was electronically submitted along with this document. The xml file is named 2024-12-23_11620012PB_seqlisting.xml, is 3225 bytes, and was created on Dec. 23, 2024.

### DESCRIPTION

### Background of the Invention

### Field of the Invention

The invention generally relates to methods of treating patients suffering from cancer using a combination of 1) an immunomodulatory agent; 2) a check point inhibitor; and 3) a cancer antigen releasing therapy (e.g. radiation). In particular, the immunomodulatory agent can be an anti-CD3 immunotoxin and the check point inhibitor inhibits the interaction of PD-1 and PD-L1.

### Background of the Invention

The interaction of the PD-L1 protein on a cell surface with the PD-1 protein on a T-cell reduces T-cell function and prevents the immune system from attacking the cell. While this is useful for the protection of normal cells, some cancer cells also express PD-L1 on their surfaces and are thus “camouflaged” as normal cells and are, unfortunately, not attacked by the immune system. Such PD-L1-bearing cancer cells are thus able to evade immune clearance. To address this problem, “check point inhibitors” (many of which are antibodies) have been developed that block the interaction of PD-L1 and PD-1, typically by binding to one or the other of these proteins and sterically inhibiting their interaction. The use of such inhibitors thus prevents PD-L1 bearing cancers from evading the immune system, resulting in recognition and destruction by the immune system.

The exemplary check point inhibitor pembrolizumab (KETRUDA®) is an FDA approved monoclonal antibody directed at the PD-1 inhibitory receptor present on T cells. Pembrolizumab has been shown to have activity against three types of cancer in humans: non-resectable melanoma, non-resectable renal cell cancer and metastatic non-small cell lung cancer. The estimated 2015 US incidence of these three diseases are; 73,870, 61,550 and 188,000 respectively. The estimated US deaths for 2015 are 9,940, 14,080 and 181,900 respectively (SEER Statistics).

In non-resectable melanoma the overall survival after treatment with KETRUDA® at 60 months was 28% and the progression free survival was 18%. While these figures represent a marked improvement over standard chemotherapy, there is still much room for improvement in survival rates.

### SUMMARY OF THE INVENTION

The present invention provides methods of killing cancer cells via a synergistic interaction between an immunomodulatory agent (e.g. an anti-CD3 immunotoxin that destroys T cells) and a check point inhibitor, in combination with the release of cancer cell antigens during homeostatic repopulation of depleted T cells.

It is an object of the invention to provide a method of treating a cancer in a subject in need thereof, comprising administering an anti-CD3 specific immunotoxin to the subject in an amount sufficient to deplete extant T-cells of the subject; providing to the subject a therapy that releases cancer antigens expressed by the cancer; and administering a check point inhibitor to the subject, wherein depletion of the extant T-cells of the patient causes repopulation and maturation of new T cells in the patient in the presence of the cancer antigens. In some aspects, the anti-CD3 specific immunotoxin is A-dmDT390-bisFv (UCHT1). In some aspects, the check point inhibitor is a monoclonal antibody inhibits the interaction of PD-1 and PD-L1 by binding to PD-L1. In further aspects, the check point inhibitor is pembrolizumab. In additional aspects, the therapy that releases cancer antigens is radiation therapy. In yet further aspects, the step of administering a check point inhibitor is performed after the step of providing release of cancer antigens. In other aspects, the step of providing release of cancer antigens is performed after the step of administering an anti-CD3 specific immunotoxin.

In additional aspects, the step of administering an anti-CD3 specific immunotoxin is performed by administering multiple doses of the an anti-CD3 specific immunotoxin on days 1, 2, 3, and 4 of treatment; the step of providing release of cancer antigens is performed on day 5 of the treatment; and the step of administering a check point inhibitor is performed on day 16 of the treatment and every 3 weeks thereafter. In further aspects, the method comprising repeating the step of providing release of cancer antigens after a period of time (e.g. after a period of days, weeks, months or years). In some aspects, the cancer is selected from the group consisting of non-resectable non-small cell lung cancer, non-resectable hepatocellular carcinoma, non-resectable head and neck squamous cell carcinoma, non-resectable colorectal cancer, non-resectable gastric carcinoma and non-resectable melanoma.

The invention also provides a method of lengthening survival time of a patient suffering from a cancer comprising: administering an anti-CD3 specific immunotoxin to the patient in an amount sufficient to deplete extant T-cells of the patient, providing a therapy that releases cancer antigens expressed by the cancer to the patient, and administering a check point inhibitor to the patient; wherein depletion of the extant T-cells of the patient causes repopulation and maturation of new T cells in the patient in the presence of the cancer antigens, thereby lengthening the survival time of the patient.

The invention also provides a method of preparing the immune system of a patient to recognize and kill metastatic and/or recurrent cancer, comprising: administering an anti-CD3 specific immunotoxin to the patient in an amount sufficient to deplete extant T-cells of the patient, providing a therapy that releases cancer antigens expressed by the cancer to the patient and administering a check point inhibitor to the patient, wherein depletion of the extant T-cells of the patient causes repopulation and maturation of new T cells in the patient in the presence of the cancer antigens, thereby preparing the immune system of a patient to recognize and kill metastatic and/or recurrent cancer.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIG. 1. Expansion of CD8 central memory T cells (TCM) induced by a 4-day treatment of RESIMMUNE® (A-dmDT390-bisFv(UCHT1) at 20 μg/kg total following homeostatic proliferation, in 8 subjects with CTCL, stage IB or IIB in a phase I trial. Tumor burdens judged by mSWAT scores varied widely from 14 to 212 yet all subjects had profound T cell depletion and subsequent homeostatic proliferation.

FIG. 2. Clinical responses in patients treated with RESIMMUNE®.

FIG. 3. Amino acid sequence of A-dmDT390-bisFv(UCHT1) (SEQ ID NO: 1).

FIG. 4. Anti-murine CD3 DT based immunotoxin, a murine analog of Resimmune, cell killing of mouse CD3 positive EL4 T cells.

FIG. 5. Anti-mouse CD3 immunotoxin delays tumor growth of GL261 cells.

### DETAILED DESCRIPTION

The present invention provides methods of killing cancer cells via a synergistic interaction between an immunomodulatory agent (e.g. an anti-CD3 immunotoxin that destroys T cells, and also destroys cancer cells that bear CD3 antigens) and a check point inhibitor, in further combination with the release of cancer cell antigens, e.g. by radiation therapy. Exemplary anti-CD3 immunotoxins are described, for example, in U.S. Pat. Nos. 7,696,338 and 8,217,158 (Neville et al.). Practice of the methods lengthens the survival time of cancer patients, and prevents and/or aids in the eradication of metastatic or recurring tumors and cancerous lesions. For example, patients remain cancer free and/or survival time is extended for at least about 6, 12, 18 or 24 months or longer. In some cases, the patients remain cancer free indefinitely.

Without being bound by theory, it is believed that when an anti-CD3 immunotoxin that destroys T cells is administered to a cancer patient, whether or not the cancer is a type which bears CD3 antigens, the immunotoxin destroys extant normal, healthy T cells expressing CD3 antigens. This results in rebound (reactive) homeostatic repopulation of new naïve CD8 central memory T cells in the treated subject. Central memory CD8 T cells generally do not persist following repopulation and the loss of the homeostatic proliferation signal. However, in the presence of tumor antigens, persistence is possible as is conversion to a central memory effector state. Thus, it is highly likely that the new central memory CD8 T cells are capable of differentiating to effector CD8 T cells which recognize and kill cancer cells of a type to which they are sensitized or trained during differentiation. According to aspects of the invention, differentiation of the new memory CD8 T cells to CD8 effector cells takes place in the presence of cancer antigens which have been i) released from tumors of the subject by a treatment such as radiation therapy, and/or ii) provided e.g. from previously preserved tumor samples; and/or iii) provided from synthetic antigen sources. Whatever the source, differentiation of the central memory CD8 T cells to effector CD8 memory T cells in the presence of the cancer antigens is highly likely to cause the effector CD8 memory T cells to specifically recognize and kill all cells bearing those antigens, such as cancer cells present in the body of the subject, cancer cells that arise later when/if the cancer recurs, etc.

Prior to conversion, central memory CD8 T cells express elevated levels of PD-1 and are thus excellent targets for treatment with check point inhibitors. Co-administration of check point inhibitors is highly likely to promotes conversion of central memory CD8 T cells to CD8 effector memory T cells. In addition, check point inhibitors directly aid in CD8 effector T cell recognition of cancer cells by blocking e.g. PD-1/PC-L1 interaction, which would otherwise camouflage the cancer cells as “normal”.

In some aspects, exposure of naïve central memory CD8 T cells to cancer antigens occurs by providing radiation therapy to the subject. The provision of radiation therapy in the treatment protocol causes the release of cancer antigens from irradiated tumors as they die and disintegrate, thus working in favor of central memory T cell conversion to T effector cells that recognize and kill tumors bearing the radiation-released antigens. When homeostatic proliferation is combined with tumor lysis and the release of tumor antigens, anti-tumor immune responses appear to persist indefinitely. Thus, the newly converted T effector cells attack tumor cells present at that time (i.e. extant tumors at other sites that were not irradiated) or in the future (e.g. if metastatic tumors develop or a cancer recurs). As a result, the combination of an anti-CD3 immunotoxin plus a check point inhibitor, and optionally/usually in combination with a tumor lysing therapy such as radiation therapy, is highly likely to result in a synergistic effect with respect to killing of cancer cells and the acquisition of long term immunity against cancer recurrence.

By “synergy” or “synergistic” we mean that the interaction of the three therapies will produces a combined effect greater than the sum of their separate effects.

Types of Agents and Therapies that are Administered

The present invention provides methods of treating cancer and/or preventing metastasis and/or recurrence of a cancer by administering a combinatorial therapy comprising 1) at least one immunomodulatory agent; 2) at least one check point inhibitor; and 3) optionally, a tumor lysing, antigen releasing therapy such as radiation therapy.

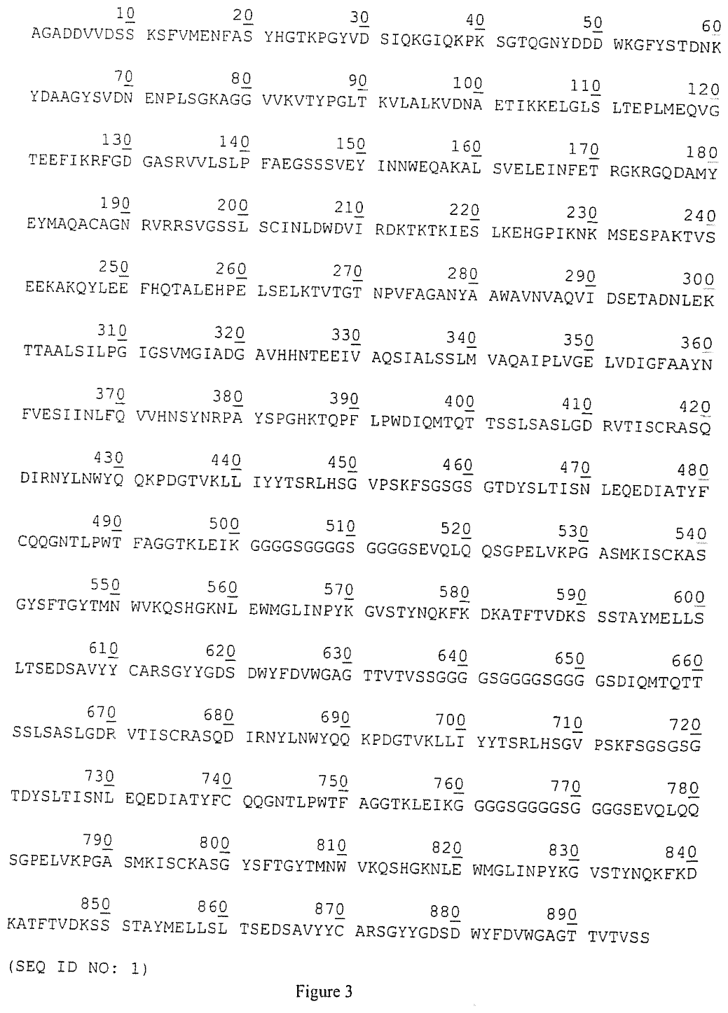

As used herein, an “immunomodulatory agent” is a compound that enhances, increases or otherwise supports or promotes the activity of the immune system. In particular, the agent increases the activity of the immune system against one or more cancers. Exemplary immunotoxin molecules are described in published U.S. patent application No. 20150166660 (the complete contents of which is hereby incorporated by reference in entirety) and are directed against the CD3 antigen of tumor cells. 20150166660 discloses that these immunotoxins surprisingly function as immunomodulatory agents. The immunotoxins were originally described in issued U.S. Pat. Nos. 7,696,338 and 8,217,158 (Neville), the complete contents of both of which are hereby incorporated by reference in entirety. Originally designed to directly kill cancer cells bearing CD3 antigens, the molecules are chimeras or fusion proteins which comprise a recombinant toxin moiety linked to an antibody moiety that is specific for binding to CD3 epitopes. The antibody moiety is responsible for binding the immunotoxin to the CD3εγ subunit of the T cell receptor complex, enabling the molecule to specifically target and bind to T-cells bearing the CD3 receptor. Once bound, the toxin moiety of the molecule enters and kills the cells. In some embodiments, the toxin moiety is, for example, a truncated diphtheria toxin (DT) moiety or pseudomonas exotoxin A (ETA) toxin moiety, and the antibody moiety comprises two single chain Fvs of and anti-CD3 antibody. The amino acid sequence of the exemplary anti-CD3 immunotoxin A-dmDT390-bisFv(UCHT1) is shown in FIG. 3 and set forth in SEQ ID NO: 1. Variants of this sequences may also be employed, e.g. variants with conservatively substituted amino acid sequences, proteolytic fragments, variants that do and do not include an amino terminal Met residue, codon optimized and/or humanized variants, etc. In addition, serine protease cleavage at e.g. furin cleavage site RVRR:SVGS (SEQ ID NO: 2; see residues 191-198 of SEQ ID NO: 1) or at other sites may occur, without disrupting the disulfide bridge between cysteines 188 and 202. Any such variant may be utilized to treat or prevent cancer as described herein, so long as immunotoxic activity is retained in the variant.

The immunotoxic agents described herein are administered to patients in a therapeutically beneficial quantity, e.g. a quantity that results in depletion of the T cell population of the patient to a level that is sufficient to elicit homeostatic repopulation of the immune system. Depletion of the T cell population refers to the destruction or killing of at least about 90 to 99% or more (e.g. 100%) of the T cells present in the subject, but in some cases killing of about 50% or more (e.g. 55, 60, 65, 70, 75 80 or 85%) may suffice.

Suitable check point inhibitors for use in the present methods include but are not limited to agents that target PD-1 and agents that target PC-L1. Exemplary anti-PD-1 agents include but are not limited to: Pembrolizumab (Keytruda®), Nivolumab (Opdivo®), Pidilizumab, etc. Exemplary anti-PD-L1 agents include but are not limited to: Atezolizumab (Tecentriq®), Avelumab (Bavencio®, MSB001071BC), Durvalumab (Imfinzi™, MED14736), AZD1775, a Wee1 G2 checkpoint serine/threonine protein kinase inhibitor, BMS-936559 (MDX-1105), MED14376, etc. In addition, in some aspects, the check point inhibitor that is used targets CTLA-4, another protein on some T cells that acts as a type of “off switch” to keep the immune system in check. Exemplary anti-CTLA-4 agents include but are not limited to: Ipilimumab (Yervoy®).

In some aspects, an antigen-releasing therapy is provided in the combinatorial protocol, e.g. an agent or therapy that lyses cancer cells and releases the antigens into circulation is administered. In some aspects, antigens are released by radiation therapy. “Radiation therapy” (“XRT”) refers to the use of high-energy radiation to shrink tumors and kill cancer cells. Radiation therapy includes the use of X-rays, gamma rays, and/or charged particles. Radiation (such as local radiation) may be delivered by a machine outside the body (external-beam radiation therapy), or it may come from radioactive material placed in the body near cancer cells (internal radiation therapy, also called brachytherapy). Alternatively, systemic radiation therapy which uses radioactive substances such as radioactive iodine may be employed. Exemplary types of radiation therapy include but are not limited to: stereotactic body radiotherapy (SBRT), stereotactic body proton therapy (RSBPT), etc.

In other aspects, antigens are instead or in addition released by agents that lyse cancer cells such as immunotoxins, for example, immunotoxins that target specific types of cancer cells such as those that target HER-2 on breast cancer cells, or antibodies that target neovasculature supplying human tumors such as anti-PSMA, etc.

### Methods

The present invention provides methods of treating cancer and/or preventing metastasis and/or recurrence of a cancer, thus lengthening survival time, disease free survival time, and cancer recurrence. The methods involve administering a combinatorial therapy comprising: I) at least one immunomodulatory agent; II) at least one check point inhibitor; and III) optionally, a tumor lysing/antigen releasing therapy such as radiation therapy. Generally the methods include a step of diagnosing a subject with cancer, metastatic cancer and/or recurrent cancer using methods known in the art.

I. Anti-CD3 immunotoxin administration: After diagnosis, the subjects are typically first treated with an immunomodulatory agent such as an anti-CD3 immunotoxin to induce reactive homeostatic proliferation and increase (amplify) the frequency (amount, level, etc.) of CD8 central memory T cells. It is especially advantageous to begin immunomodulation as soon after diagnosis as possible because the benefits of the treatment are typically not observed for at least weeks, or months, or even years after the treatment, and it is desirable for the benefits to accrue as soon as possible.

Doses of immunotoxin may be varied according to parameters that are understood by those of skill in the art, e.g. by a skilled medical practitioner. Recommended doses and particular protocols for administration may be established during clinical trials. The amount may vary based on e.g. the body weight, gender, age, overall condition, etc. of the patient, and/or on the type and stage of disease, and whether or not other therapeutic agents are being administered, etc. Generally, the total amount administered during a round of chemotherapy (scheduled to take place over a period of several days e.g. a period of 4 days, will range from about 5 to about 60 μg/kg of body weight, e.g. the amount that is administered may be, for example, about 5, 10, 15, 20, 25, 30, 35, 40, 45, 50, 55 or 60 μg/kg of body weight. Typically, a total of about 20 μg/kg of body weight is administered over e.g. 4 days. This amount is usually administered at multiple times or sessions during a single day of e.g. about 0.5 to 5 (e.g. about 0.5, 1.0, 1.5, 2.0, 2.5, 3.0, 3.5, 4.0, 4.5 or 5.0 μg/kg of body weight per session, with e.g. 1-6 (e.g. 1, 2, 3, 4, 5, or 6) sessions per day, and usually about 2 sessions per day. The number of days or weeks for which the treatment proceeds may also vary (e.g. from about 2 to about 10 or more days, e.g. about 2, 3, 4, 5, 6, 7, 8, 9, or 10 days) depending on the factors which impact dosage and timing. For example, the total amount of the exemplary anti-CD3 agent A-dmDT390-bisFv(UCHT1) that is administered is typically about 20 mcg/kg, a dose shown to induce homeostatic proliferation (providing anti-DT titers are less than 22 mcg/ml). This dose is typically administered as 8 equally divided doses, each of which is administered twice daily for a period of 4 days, i.e. days 1˜4 of treatment. A-dmDT390-bisFv(UCHT1) induced T cell depletion is usually done during a single 4-day course, since anti-DT antibody titers rise after day 5 and interfere with subsequent depletions until titers decline, e.g. 3 to 12 months later. In order to address this, in some aspects, deimmunized truncated toxin sequences are employed so that the course of immunotoxin treatment is extended, e.g. to 5 or 6 days, or to a week or more.

Typically, a 4 day treatment with the anti-CD3 agent (twice daily) causes a 2-3 log depletion of normal T cells and induces reactive homeostatic proliferation. The level of circulating T cells, including mature CD3 T cells and central memory and effector memory CD8 T cells, may be monitored by methods known in the art to optimize treatment and achieve treatment goals. Likewise, the levels of other immune system components such as antibodies to the toxin moiety of the immunotoxin, or to other treatment or vehicle components, and/or to tumor antigens, etc. as deemed useful. Generally, T cell replication increases the frequency of CD8 central memory T cells by 10-20 fold over pretreatment levels by days 15-38. These cells display the activated markers and functions CD45RA^{-/low}, CD3, CD27 and CD8.

II. Tumor antigen release. Administration of anti-CD3 agents is coordinated with other therapies that release cancer antigens to provide an opportunity for repopulating T cells to be “trained”. Therefore, the present methods also include a step of killing tumor cells in a manner that releases tumor antigens, or of otherwise exposing developing effector T cells to cancer antigens, to facilitate the development of immune cell memory with respect to the cancer antigens. In general, tumor cell antigens are released or otherwise provided to the subject using one or more suitable agents or therapies about 5-6 days after administration of the anti-CD3 immunotoxin. The antigen release therapy that is used should not damage local lymphoid structure and function.

If the tumor lysing therapy is local radiation, the amount that is delivered is chosen by the radiation oncologist and is typically delivered as a single fraction on e.g. day 5 of treatment (i.e. 1-2 days after 4 days of anti-CD3 immunotoxin administration) and may vary between 3-20 Gy e.g. about 3, 4, 5, 6, 7, 8, 9, 10, 11, 12, 13, 14, 15, 16, 17, 18, 19 or 20 Gy. Many factors are considered when selecting a dose, including whether the patient is receiving chemotherapy, co-morbidities, whether radiation therapy is being administered before or after surgery, and the degree of success of surgery, etc. Higher doses of radiation (e.g. in the range of 10-20 Gy per exposure) may increase the response rate of lesions outside of the radiation field and thus provide a more marked effect with respect to immunomodulation.

As stated above, other antigen releasing therapies may also be employed instead of or in addition to radiation. For example, if the cancer cells do bear CD3 antigens, then the anti-CD3 agent will, fortuitously, also cause killing of cancer cells and antigen release.

Other sources of antigens are, for example, antigen preparations prepared from tumor or biopsy samples taken from the subject. Such samples may be stored and portions administered together with or in conjunction (association) with the methods described herein at suitable time intervals, e.g. about 1-2 days after the 4-days of anti-CD3 agent administration.

The step of releasing cancer antigens is generally carried out early in treatment, and may be sufficient to put the immune system in condition to monitor, recognize and eradicate new tumors shortly after recurrence without further treatment. However, in other aspects, antigen-releasing therapy may be reapplied later during the course of treatment in order to further boost or prime the immune response, analogous to a vaccination protocol. This may be readily accomplished if the cancer recurs since a treatment that releases antigen can be administered at that time. However, if no visible or detectable recurrence is present, it is possible to effect boosting by administering tumor cells or antigen-bearing fragments thereof from the original tumors that have been preserved for the purpose. In this case, the cells or fragments can be administered e.g. 3-6 months after the initial treatment as a “booster”, and/or at longer intervals (e.g. yearly) thereafter, if desired. This facilitates the treatment of non-irradiated and/or developing metastatic and/or recurring cancer ahead of time by augmenting the patient's natural ability to conduct immune surveillance on an ongoing basis and fight the development of tumors. Such radiation doses and protocols are similar to those described above for a first treatment.

### III. Check Point Inhibitor Administration

Administration of the check point inhibitor is typically performed after the antigen-releasing therapy, e.g. about 7-10 days thereafter, e.g. on or about day 16 of treatment (with days 1-4 of treatment being the days of administration of the anti-CD3 immunotoxin). Thereafter, the check point inhibitor is administered about every three weeks for a period of time of several (e.g. 1-6) months and/or until cancer cells are no longer detectable in the subject or until side effects from the checkpoint inhibitor cause discontinuation of the checkpoint inhibitor.

The dose of check point inhibitor is generally in the range of that prescribed by the FDA for new drug approval or for investigational drugs from the FDA approved Investigational New Drug policy. In one aspect, the doses are reduced from clinical indications during the phase I portion of the drug combinational trial due to the influence of the combination drug synergistic effects of an anti-CD3 immunotoxin and checkpoint inhibitors. For example, the one or more check point inhibitors are administered e.g. at doses of from about 0.1 to about 100 mg/kg, or from about 1 to about 75 mg/kg, or about 5 to about 50 mg/kg per dose that is administered. Administration is generally at the time of or within the e.g. 4 day period during which the anti-CD3 agent is administered. One administration may be sufficient, or the total dose may be divided into small doses, and/or the dose may be repeated e.g. weekly, monthly, etc. as need to achieve the desired effect.

A course of treatment may be repeated as needed throughout the patient's lifetime, especially if there is a recurrence of the cancer. However, for such repetitions of treatment, in general it is not necessary to repeat administration of the anti-CD3 immunomodulator, but only the local tumor radiation (as described above), with or without administration of a check point inhibitor.

The methods of the invention are carried out by administering compositions which include the active agents described herein (or in some cases, nucleic acid sequences encoding them) and a pharmacologically suitable (physiologically compatible) carrier. The preparation of such compositions is known to those of skill in the art. Typically, such compositions are prepared either as liquid solutions or suspensions. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like.

Generally, the anti-CD3 agent and the check point inhibitor (and/or the antigen releasing agent, if the latter is not radiation) are administered “together” (in combination) as separate components of a cancer treatment protocol. As such, each agent is usually administered as a separate composition. Administration may or may not be performed so that one or more agents are present in the blood stream of the treated subject at the same time. However, the antigen releasing therapy is preferably performed after the T-cell population is reduced, and before or during repopulation. It is generally desirable to have check point inhibitor present during repopulation and conversion of memory T cells, and thereafter to augment the activity of effector T cells. Administration is systemic.

While the agents described herein are generally administered as separate compositions, this need not always be the case. Compositions comprising one or more anti-CD3 agents, and/or one or more check point inhibitors and/or one or more antigen releasing agents are also encompassed.

Those of skill in the art are familiar with the administration of chemotherapeutic agents, and the compositions (preparations) may be administered by any of the many suitable means which are known, including but not limited to: by injection, inhalation, orally, intravaginally, intranasally, topically, as eye drops, via sprays, by intra-tumor injection, by implantation of a device or composition that releases the agent, etc. Generally, the mode of administration is intravenous or subcutaneous.

In addition, the compositions may be administered in conjunction or in association with other treatment modalities such as substances that boost the immune system (e.g. tuberculosis bacilli or portions thereof), various other anti-tumor/anti-cancer chemotherapeutic agents (e.g. platinum drugs such as cis-platinum, methotrexate, etc.), pain medication, anti-nausea medication, anti-allergy agents (e.g. anti-histamines), nutrition supplements, anti-depressants, and the like. Likewise, surgery may also be incorporated as part of the overall treatment, e.g. to resect one or more tumors before the present methods are begun, or at any suitable time thereafter. Additional combinable cancer treatments are described in more detail below.

The agents described herein may be administered at any desired time after diagnosis of a cancer, and by any suitable protocol or schedule. They may be administered before, after or at the same time as other anticancer agent or therapy. For example, they may be administered prior to the commencement of treatment with other cytotoxic agents or therapies or surgery, and/or together with them, or after other cytotoxic agents, therapies and/or surgeries have been administered or performed, e.g. several days or weeks afterwards. If administered “together” with another treatment modality, they may be provided separately e.g. in separate compositions that are administered within a short time of each other such as within minutes, hours, days or weeks, or using a single composition that contains at least one (i.e. one or more) agent that is an anti-CD3 agent, a check point inhibitor, or an antigen releasing therapy, if not radiation), and one or more than one other anti-cancer agent, etc.

Types of Cancer that are Treated

In some aspects, subjects who are identified as suitable for treatment using the methods of the invention are those who are diagnosed as suffering from a cancer in which the cancer cells do not bear surface CD3 epitopes i.e. CD3 epitopes are not present on (are absent from) the surface of the cancer cells. In such aspects, if an anti-CD3 immunotoxin is used as the immunomodulatory agent, it functions largely as an immunomodulatory agent, exhibiting no direct killing of cancer cells. Cancers which do not bear surface CD3 epitopes include any non-T cell leukemia or lymphoma (i.e. any cancer that is not a T cell leukemia or lymphoma; acute myeloid leukemia (AML); adrenocortical carcinoma; atypical teratoid/rhabdoid tumors; central nervous system cancers; basal cell carcinoma (e.g. nonmelanoma); bile duct cancer; bladder cancer; extrahepatic bladder cancer; bone cancers (e.g. Ewing sarcoma family of tumors, osteosarcoma and malignant fibrous histiocytoma; brain stem glioma; brain tumors (e.g. astrocytomas, brain and spinal cord tumors, CNS atypical teratoid/rhabdoid tumor, CNS embryonal tumors, CNS germ cell tumors, etc.); craniopharyngioma, ependymom; breast cancer; bronchial tumors, Burkitt lymphoma gastrointestinal tumors; cardiac (heart) tumors; cervical cancer; chordoma; chronic lymphocytic leukemia (CLL); chronic myelogenous leukemia (CML); chronic myeloproliferative disorder; colon cancer; colorectal cancer; craniopharyngioma; extrahepatic bile duct tumors; ductal carcinoma in situ (DCIS); embryonal tumors; endometrial cancer; esophageal cancer; esthesioneuroblastoma; Ewing sarcoma; extracranial germ cell tumor; extragonadal germ cell tumor; eye cancers (intraocular melanoma, retinoblastoma); fibrous histiocytoma of bone; osteosarcoma; gallbladder cancer; gastric (stomach) cancer; gastrointestinal carcinoid tumor; gastrointestinal stromal tumors (GIST); gestational trophoblastic tumor; glioma; hairy cell leukemia; head and neck cancer; heart cancer; hepatocellular (liver) cancer; hypopharyngeal cancer; intraocular melanoma; islet cell tumors; pancreatic neuroendocrine tumors; kidney (e.g. renal cell and Wilms tumor); langerhans cell histiocytosis; laryngeal cancer; leukemia; liver cancer (primary); lobular carcinoma in situ (LCIS); lung cancer (non-small cell, small cell); lymphomas; Waldenström macroglobulinemia; male breast cancer; malignant mesothelioma, metastatic squamous neck cancer with occult primary midline tract carcinoma involving NUT gene; mouth cancer; multiple endocrine neoplasia syndromes; myelodysplastic syndromes; myelodysplastic/myeloproliferative neoplasms; Chronic Myelogenous Leukemia (CML); Acute Myeloid Leukemia (AML); multiple myeloma; chronic myeloproliferative disorders; nasal cavity and paranasal sinus cancer; nasopharyngeal cancer; neuroblastoma; non-Hodgkin lymphoma; oral cancer; oral cavity cancer; lip and oropharyngeal cancer; osteosarcoma and malignant fibrous histiocytoma of bone; ovarian cancer; pancreatic cancer; pancreatic neuroendocrine tumors (Islet Cell tumors); papillomatosis; paraganglioma; parathyroid cancer; penile cancer; pharyngeal cancer; neochromocytoma; pituitary tumor; plasma cell neoplasm/multiple myeloma; pleuropulmonary blastoma; prostate cancer; rectal cancer; renal cell (kidney) cancer; salivary gland cancer; sarcomas (Ewing, Kaposi, osteosarcoma, rhabdomyosarcoma, soft tissue, uterine); skin cancers (melanoma, Merkel cell carcinoma, nonmelanoma); small intestine cancer; squamous cell carcinoma; squamous neck cancer; metastatic stomach (gastric) cancer; testicular cancer; throat cancer; thymoma and thymic carcinoma; thyroid cancer; transitional cell cancer of the renal pelvis and ureter; trophoblastic tumor, gestational; urethral cancer; uterine cancer, endometrial cancer; uterine sarcoma; vaginal cancer; vulvar cancer; Waldenström macroglobulinemia; Wilms tumor; nasal cavity and paranasal sinus cancer; nasopharyngeal cancer; neuroblastoma; non-small cell lung cancer; melanoma of the skin, Hodgkin lymphoma, and metastases and recurrences thereof.

### Other Combinable Therapies

In some aspects, other toxic agents and/or other therapies are also used for treatment, e.g. to kill the cancer cells outright, to cause tumor shrinkage, etc. e.g. for short-term, front line therapy. Thus, one or more other anti-cancer agents or anti-cancer modalities or therapies may also be administered, examples of which include but are not limited to: cytotoxic immunotoxins targeting the specific tumor or blood vessels growing into the tumor, cytotoxic antineoplastic drugs such as alkylating agents cisplatin, carboplatin, oroxaliplatin; anti-metabolites which masquerade as purines (e.g. azathioprine, mercaptopurine) or pyrimidines; plant alkaloids and terpenoids, e.g. vinca alkaloids such as vincristine, vinblastine, vinorelbine, vindesine; podophyllotoxin, etoposide and teniposide; taxanes such as paclitaxel; type I topoisomerase inhibitors including the camptothecins irinotecan and topotecan, and type II topoisomerase inhibitors such as amsacrine, etoposide, etoposide phosphate, and teniposide; and cytotoxic antibiotics such as actinomycin, anthracyclines, doxorubicin, daunorubicin, valrubicin idarubicin, epirubicin, bleomycin, plicamycin and mitomycin; gene therapy (e.g. to deliver a nucleic acid encoding an anti-cancer agent to a tumor), surgery/resection of tumors; hormonal therapy; administration of angiogenesis inhibitors; administration of other immunomodulating agents or therapies (e.g. allogeneic or autologous hematopoietic stem cell transplantation (the patient that is treated may or may not be a transplant patient); by radiation therapy via external beam radiotherapy (EBRT) or internally via brachytherapy, electrochemotherapy; ultraviolet (UV) light therapy; etc.

The examples presented below are intended to illustrate various exemplary aspects of the invention but should not be interpreted so as to limit the invention in any way.

### Examples

EXAMPLE 1. Adaptive clinical trial design of the A-dmDT390-bisFv(UCHT1) Fusion Protein (RESIMMUNE®) in Combination with Ionizing Radiation and anti-PD-L1 for the treatment of non-resectable non-small cell lung cancer, non-resectable hepatocellular carcinoma, non-resectable head and neck squamous cell carcinoma, non-resectable colorectal cancer, non-resectable gastric carcinoma and non-resectable melanoma.

An Adaptive clinical trial design is defined as a study that includes a prospectively planned opportunity for modification of one or more specified aspects of the study design and hypotheses based on analysis of data (usually interim data) from subjects in the study. Analyses of the accumulating study data are performed at prospectively planned timepoints within the study, can be performed in a fully blinded manner or in an unblinded manner, and can occur with or without formal statistical hypothesis testing.

This trial begins with a dose escalation study to evaluate the safety of the combination therapy in various solid cancers. All enrolled subjects from each pre-planned combination dose level are observed for DLT within 28 days of the first dose administered. Once a planned number of subjects from each dose (3 subjects first from 3+3 design) pass the 28 days DLT window, that dose level is cleared and the study moves to the next dose level or expansion phase. Since the end point is for safety, not efficacy, more tumor types with unmet needs are included and there is no need to wait till an efficacy point to move to the next dose level, so that the study has faster enrollment and a shorter time to move to the expansion. Anti-PD-L1 dose escalation in 3 phase I trials that has reached 1 mg/kg is the low dose progressing to 2.5 mg/kg and finally 5 mg/kg, (3 subjects first from 3+3 design). The dose of Resimmune® is kept constant at 20 □g/kg total over 4 days given twice daily as this dose induces profound T cell depletion and subsequent homeostatic proliferation and generation of a 20-20-fold increase in TCM-like cells. All enrolled subjects from each pre-planned combination dose level are observed for DLT within 28 days of the first dose administered. Once a planned number of subjects from each dose (3 subjects first from 3+3 design) pass the 28 days DLT window, that dose level is cleared and the study moves to the next dose level or expansion phase. Since the end point is for safety, not efficacy, more tumor types with unmet needs are included and there is no need to wait till an efficacy point to move to the next dose level, so that the study has faster enrollment and a shorter time to move to the expansion. The dose of radiation, preferably SBRT, is chosen by the radiation oncologist to be palliative rather than curative and in the range of 14 to 24 Gy depending on the metastatic lesion size. In the dose expansion phase where efficacy is evaluated there is an option of adding a second arm that is minus Resimmune to establish the size of the benefit of the Resimmune combination. As the numbers become available statistics are used to predict the optimal cohort size

A minimum benefit of the combination of Resimmune® with anti-PD-L1 over anti-PD-L1 alone is 135% and increased benefits up to 250% are observed This increase is likely because the combination therapy has the capacity to increase the presentable tumor neoantigens and eliminate critical T cell suppressor mechanisms operating within the tumor microenvonment.

Exclusions: Subjects having hepatocellular carcinoma must be shown to be free of replicating hepatic viruses; Subjects with a prior history of cardiac disease are excluded. See INVESTIGATOR'S BROCHURE: Use of A-dmDT390-bisFv(UCHT1) in the Treatment of Mycosis Fungoides

In non-resectable melanoma, the overall survival after treatment with the anti-PD-1 check point inhibitor KETRUDA® at 60 months was 28% and the progression free survival was 18%. The next therapeutic challenge is to ascertain why some patients respond and others do not so that the response rate and overall survival can be increased.

It has been shown in clinical studies treating cancers with checkpoint inhibitors that the number of tumor somatic mutations present strongly correlates with clinical success even though tumor-infiltrating lymphocytes (TILs) could recognize tumors with relatively low numbers of somatic mutations. This could be a quantitative problem with low numbers of somatic mutations resulting in too few TILs or TILs with insufficient T cell activation. In mouse tumor studies, agents that induce homeostatic proliferation augment the anti-tumor activity of adoptively transferred T cells. PD-1/PD-L1 blockade promotes effector function of central memory T cells presumably because central memory T cells express elevated levels of PD-1.

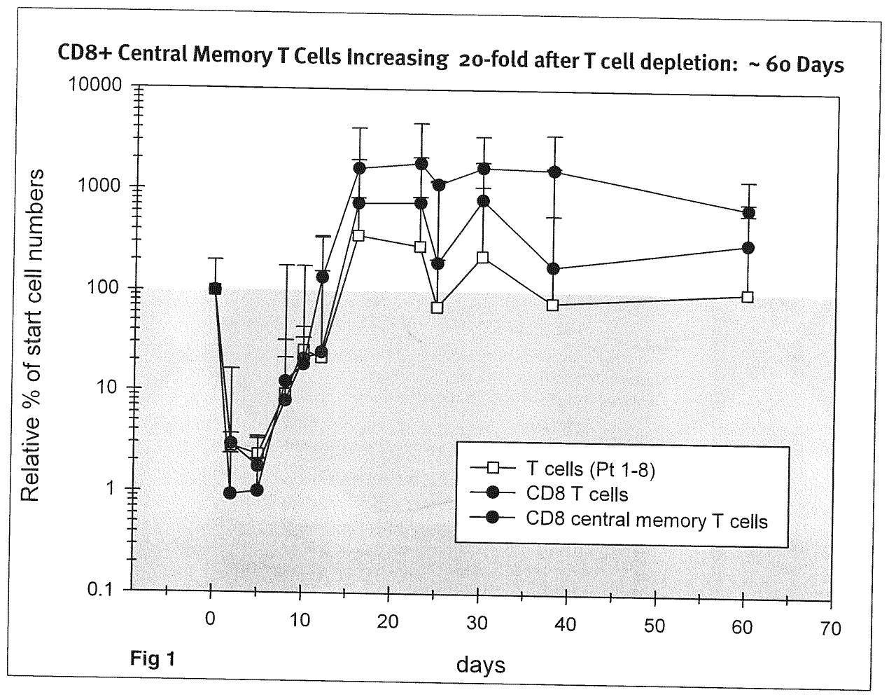

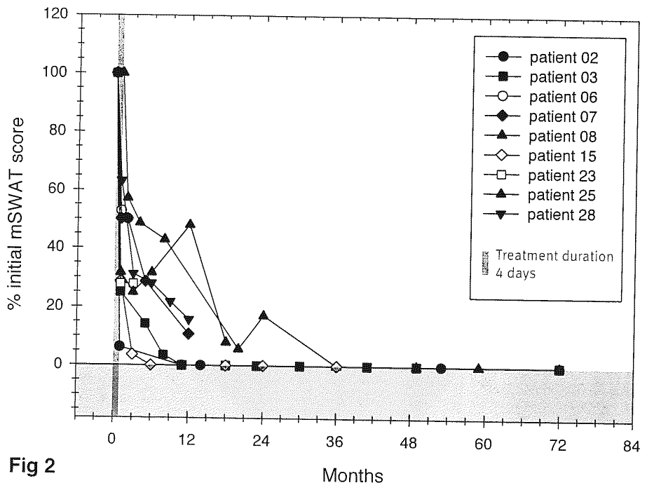

RESIMMUNE® induces T cell depletion and subsequent homeostatic central T cell repopulation in patients with cutaneous T cell lymphoma, resulting in a 20-fold increase in circulating central memory T cells from day 16 to day 140 (FIG. 1, which shows data up to 60 days). These central memory T cells can convert to effector memory T cells in the presence of tumor antigens, and the 20-fold increase provides a quantitative increase in the number of tumor specific central memory T cells and thus an increased probability of conversion to effector memory T cells, resulting in immune mediated tumor suppression. This appears to be the mechanism of the immunomodulation that results in long-term complete responses seen with RESIMMUNE® therapy, but not observed with conventional therapy (see FIG. 2).

A check point inhibitor aids in this process through PD-1/PD-L1 blockade. This treatment protocol is especially effective in melanoma patients, since melanoma has a high somatic mutation rate. However, the treatment protocol is also successful in other cancers with lower somatic mutation rates, e.g. hepatocellular carcinoma cancer) because of the compensatory boost in central memory T cell numbers (afforded by RESIMMUNE™) that can convert to effector T cells (via antigen exposure as a result of radiation therapy).

In an exemplary aspect, patients with unresectable, stage IV cancers are treated with a combination of the anti-CD3 agent RESIMMUNE®, anti-PD-L1 and palliative radiation therapy. The palliative radiation therapy acts as an immune adjuvant by releasing melanoma antigens and causing infiltration of dendritic cells and T-cells into tumors (Table 1).

| | Timeline | | | |
|---|---|---|---|---|
| Parameter | Days 1-4 | Day 5 | Day 16 | >90 Days |
| Treatment | RESIMMUNE ® | Radiation: | Start | Imaging |
| or | administered | single | anti-PD-Ll | |
| Procedure | once daily | palliative | administer | |
| | | dose | thereafter | |
| | | | once every | |
| | | | 3 weeks | |
| Outcome, | T-cell depletion | T-cell | T-cell | Immunity |
| Ongoing | | proliferation | proliferation | Tumor |
| events | | Clonal | Clonal | regression |
| | | expansion | expansion | |
| | | Antigen | | |
| | | presenting cell | | |
| | | infiltration | | |

A secondary objective is to use high-throughput deep sequencing of the human TCR V-β region to better characterize the expansion and clonality of the peripheral T-cell repertoire during the therapy. Immunotherapy-induced changes in the T-cell repertoire are correlated with peripheral T-cell homeostatic proliferation/activation and immune suppressor cell percentages/phenotype (T regulatory cells and IL-17⁺ T-cells). These changes are compared to responses obtained with anti-PD-L1 or anti-PD-1 treatment without RESIMMUNE® and radiation and serve to provide proof of principle for the combination therapy. Combining the characterization of peripheral TCR clonality with biomarkers of efficacy is a valuable method to track cancer-specific T-cells in the peripheral blood when the predominant stimulating antigen is unknown and thus, is used as a predictive biomarker to assess melanoma patients' response or resistance to immunotherapies.

EXAMPLE 2. A mouse model to demonstrate the effect of anti-CD3 immunotoxin on tumor growth that can probe the synergism between checkpoint inhibitors and anti-CD3 immunotoxins.

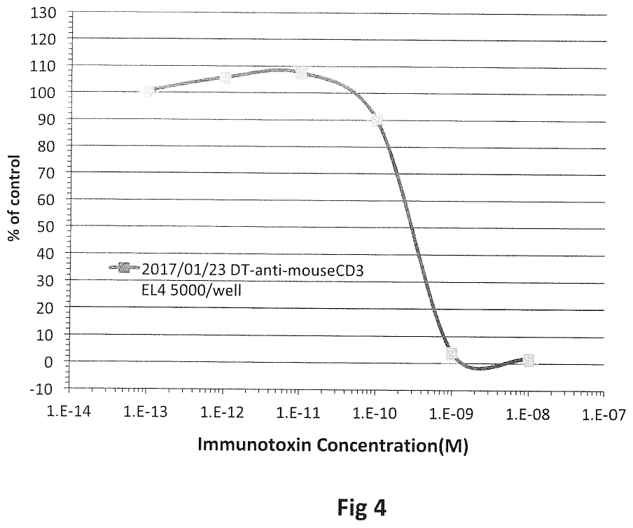

In order to demonstrate the anti-tumor effect of the anti-CD3 immunotoxin, we have developed an anti-mouse CD3 immunotoxin which is an analog of the anti-human CD3 immunotoxin, Resimmune®. The anti-mouse CD3 immunotoxin has the same structure as its human counterpart and is also specific for the epsilon chain of the CD3 complex. The immunotoxin is capable of killing efficiently mouse CD3 positive T cells (FIG. 4).

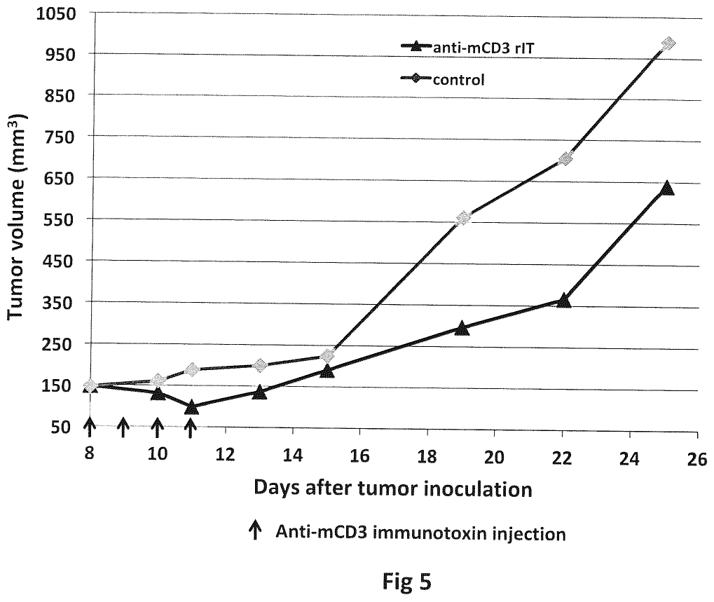

In a solid tumor model, we used immunocompetent mice C57BL/C and a mouse glioma cell line, GL261. The cell line does not express mouse CD3, and therefore was not sensitive to the anti-mouse CD3 immunotoxin. GL261 cells (5×10⁵/mouse) were inoculated subcutaneously into the right flanks of C57BL/C mice and tumor xenografts were allowed to grow to about 150 mm³ in size before treatment with the anti-mouse CD3 immunotoxin or PBS buffer. Tumor-bearing C57BL/C mice were treated though tail vein injection (one injection per day for four days) with 0.1 ml PBS buffer as control or 0.1 ml anti-mouse CD3 immunotoxin in PBS (40 μg/kg). As shown in FIG. 5, the anti-mouse CD3 immunotoxin can significantly delay tumor growth.

Since the anti-mouse CD3 immunotoxin has no direct effect to the growth of GL261cells, the anti-tumor activity to GL261xenografts is mostly due to the immunomodulation effect as seen in Resimmune®-CTCL clinical trials. Without being bound by theory, it is believed that T cell depletion by anti-CD3 immunotoxin leads to the rapid expansion of cytotoxic CD8+ T cells through homeostatic proliferation and is the cause for the anti-tumor activity. It is known that tumors express checkpoint molecules, such as PD-L1 to escape T cell attack, therefore, when an anti-CD3 immunotoxin is combined with a checkpoint inhibitor, such as an anti-PD-1 or PD-L1 antibody, a more profound and long-lasting tumor-inhibition effect is achieved in mouse tumor models and in human cancer therapy.

A checkpoint inhibitor, such as an anti-murine anti-PD-1 or PD-L1 antibody, in combination with anti-murine anti-CD3 immunotoxin increases the overall response rate by at least 135% over the non-treatment control when judged by inhibition of tumor growth or tumor regression and reaches levels of 250% over the non-treatment-control.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of treating a cancer in a subject in need thereof, comprising
administering an anti-CD3 specific immunotoxin to the subject in an amount sufficient to deplete extant T-cells of the subject,
providing to the subject a therapy that releases cancer antigens expressed by the cancer, and
administering a check point inhibitor to the subject,
wherein depletion of the extant T-cells of the patient causes repopulation and maturation of new T cells in the patient in the presence of the cancer antigens.

2. The methods of claim 1, wherein the anti-CD3 specific immunotoxin is A-dmDT390-bisFv(UCHT1).

3. The method of claim 1, wherein the check point inhibitor inhibits the interaction of PD-1 and PD-L1.

4. The method of claim 3, wherein the check point inhibitor is an anti-PD-L1 monoclonal antibody or an anti-PD-1 monoclonal antibody.

5. The method of claim 1, wherein the therapy that releases cancer antigens is radiation therapy.

6. The method of claim 1, wherein
the step of administering a check point inhibitor is performed after the step of providing release of anti-tumor antigens.

7. The method of claim 1, wherein
the step of providing release of anti-tumor antigens is performed after the step of administering an anti-CD3 specific immunotoxin; and
the step of administering a check point inhibitor is performed after the step of providing release of anti-tumor antigens.

8. The method of claim 1, wherein
the step of administering an anti-CD3 specific immunotoxin is performed by administering multiple doses of the an anti-CD3 specific immunotoxin on days 1, 2, 3, and 4 of treatment;
the step of providing release of anti-tumor antigens is performed on day 5 of the treatment; and
the step of administering a check point inhibitor is performed on day 16 of the treatment and every 3 weeks thereafter.

9. The method of claim 1, further comprising
repeating the step of providing after a period of time.

10. The method of claim 1, wherein the cancer is selected from the group consisting of non-resectable non-small cell lung cancer, non-resectable hepatocellular carcinoma, non-resectable head and neck squamous cell carcinoma, non-resectable gastric carcinoma, non-resectable colorectal cancer and non-resectable melanoma.

11. A method of lengthening survival time of a patient suffering from a cancer comprising
administering an anti-CD3 specific immunotoxin to the patient in an amount sufficient to deplete extant T-cells of the patient,
providing a therapy that releases cancer antigens expressed by the cancer to the patient, and
administering a check point inhibitor to the patient,
wherein depletion of the extant T-cells of the patient causes repopulation and maturation of new T cells in the patient in the presence of the cancer antigens, thereby lengthening the survival time of the patient.

12. A method of increasing the overall response rate of a subject suffering from a metastatic and or recurrent cancer by preparing the immune system of a patient to recognize and kill metastatic and/or recurrent cancer, comprising
administering an anti-CD3 specific immunotoxin to the patient in an amount sufficient to deplete extant T-cells of the patient,
administering a check point inhibitor to the patient, and
providing radiation therapy to one metastatic lesion that releases cancer antigens expressed by the cancer to the patient,
wherein depletion of the extant T-cells of the patient causes repopulation and maturation of new T cells in the patient in the presence of the cancer antigens, thereby preparing the immune system of a patient to recognize and kill metastatic and/or recurrent cancer by a factor of at least 135% over the administration of the check point inhibitor alone without anti-CD3 specific immunotoxin and without radiation therapy to one metastatic lesion.


SEQUENCE LISTING (1 sequence)

SEQ ID NO: 1 - protein, 896 aa, synthetic construct
  REGION 1..896: note = Synthetic recombinant toxin molecule
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATFTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATFTVDKSSSTAYMELLSLTSEDSAVYYCARSGYYGDSDWYFDVWGAGTTVTVSS